(19) United States
(12) Patent Application Publication
(10) Pub. No.: US 2021/0125702 A1
(43) Pub. Date: Apr. 29, 2021

(54) STRESS MANAGEMENT IN CLINICAL SETTINGS

(71) Applicant: SHAFTESBURY INC., Toronto (CA)

(72) Inventors: Edward W. BIGGS, Toronto (CA); Brianna LOWE, Toronto (CA); Justin Robert CAGUIAT, Toronto (CA); Naimul Mefraz KHAN, Toronto (CA); Nabila Miriam ABRAHAM, Mississauga (CA); Muhammad Rehman ZAFAR, Toronto (CA); Syeda Suha Shee RABBANI, Toronto (CA); Zeeshan AHMAD, Mississauga (CA); Mihai Constantin ALBU, Dundas (CA); Jacky ZHANG, Markham (CA)

(21) Appl. No.: 16/663,223

(22) Filed: Oct. 24, 2019

(51) Int. Cl. G16H 20/70; G16H 10/20

(52) U.S. Cl. CPC G16H 20/70; G16H 10/20

(57) ABSTRACT

An example of an apparatus including an output device to provide content to a user is provided. The content is to distract the user from an event. The apparatus further includes a sensor to collect user data in response to the content generated by the output device. The user data is to provide information about a state of the user. In addition, the apparatus includes a communications interface connected to the sensor. The communication interface is to transmit the user data to an analyzer. The analyzer is to determine a stress level of the user based on the user data. The communications interface is further to receive the stress level of the user data from the analyzer. The apparatus also includes a content selection engine to control the content provided to the user based on the stress level.

### BACKGROUND

Clinical settings are often used to provide healthcare to individuals in a clinical care environment. For example, individuals may use such settings to undergo various procedures, such as diagnosis of a medical condition, or treatment of a medical condition. The complexity of the procedures carried out in clinical settings may widely range from a relatively simple procedure, such as the measurement of a temperature reading of the patient, to a very complicated procedure, such as a surgical procedure that may result in a lengthy stay for the patient in the clinical setting. Accordingly, some medical procedures may last several hours, days, or even weeks in a clinical setting such as a hospital. During longer stays in the clinical setting, anxiety and stress in a patient may have negative effects on the health of the patient resulting in potential complications. The negative effects may generally be controlled using medications or therapy sessions.

### BRIEF DESCRIPTION OF THE DRAWINGS

Reference will now be made, by way of example only, to the accompanying drawings in which:

FIG. 1 is a block diagram of an example apparatus to manage stress in a clinical setting;

FIG. 2 is a block diagram of another example apparatus to manage stress in a clinical setting;

FIG. 3 is a perspective view of another example apparatus to manage stress in a clinical setting mounted on a user;

FIG. 4 is a block diagram of an example system to manage stress in a clinical setting mounted on a user;

FIG. 5 is a block diagram of an example server to process data from an apparatus to determine and provide a stress level of a user; and

FIG. 6 is a flowchart of an example method of managing stress in a clinical setting.

### DETAILED DESCRIPTION

Waiting for a medical procedure in a clinical setting, such as a hospital, may generate feelings of stress and anxiety in patients, especially for small children. Stress and anxiety has been determined to have a negative effect on patients in such clinical care situations. Increased stress may create a variety of problems within the human body, which may be especially detrimental to the immune system in addition to the neuroendocrine and metabolic systems. Furthermore, increased stress may be linked to adverse health outcomes such as prolonged recovery periods from a medical procedure. In some cases, these adverse effects may include resistance to treatment, nightmares, and anxiety. Children may be additionally susceptible to experiencing the physical impact of stress and anxiety when in a clinical setting such as a hospital, whether it is in waiting rooms or a preoperative setting.

Accordingly, long term hospitalization as well as any state of disease or poor health may be stressful for children and may trigger multiple physiological mechanisms that may result in the disruption of normal development and have long term consequences. In addition to these physiological changes, stress and anxiety experienced by children in a clinical setting may be linked to adverse outcomes, such as increased recovery periods, resistance to treatment and nightmares.

An apparatus, system and method is provided to relieve stress prior to, during, and/or after medical procedures, such as surgical operations. The management of stress may increase the ability of patients, especially children, to heal faster. In addition to the management of stress in the patient, a less stressed patient, such as a child patient, in a relaxed and calm state may provide additional benefits to individuals close to the patent by ameliorating the anxiety of an attending caregiver or family member. In particular, engaging a patient by providing entertainment was found to alleviate the signs of stress in the clinical setting. The type of entertainment provided is not particularly limited and may include animations, toys, published material, video content, such as a movie or television programming, or audio content, such as music. It is to be appreciated that content that is more immersive, such as a virtual reality content may have a stronger positive effect on patients in clinical settings. Accordingly, more immersive content may be more efficient to manage and relieve stress.

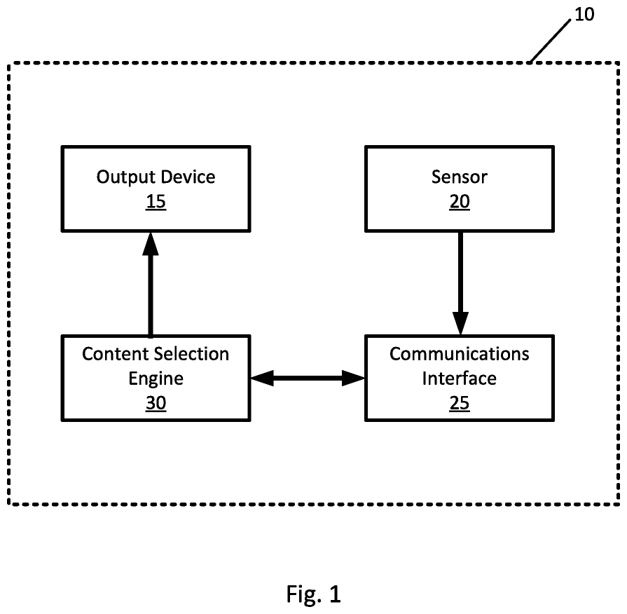

Referring to FIG. 1, a schematic representation of an apparatus to manage stress in a clinical setting is generally shown at 10. The apparatus 10 may include additional components, such as various additional interfaces and/or input/output devices such as displays to interact with the user of the apparatus 10, such as to change a setting or otherwise to reprogram the apparatus 10 locally. The apparatus 10 is to provide content, such a virtual reality content, to a user or patient, and to control and/or modify the content based on a stress level of the patient determined by analyzing data from the user. In some examples, the content may be modified in response to a reaction by the user or patient, such reducing the level of stimulation when an increase in stress and anxiety is detected, or increasing the level of stimulation when an increase in the level of user boredom is detected. In the present example, the apparatus 10 includes an output device 15, a sensor 20, a communications interface 25, and a content selection engine 30.

The output device 15 is to provide content to a user, such as a patient in a clinical setting having feelings of stress or anxiety. It is to be appreciated that the patient may not be anxious or stressed in some examples and be using the content for entertainment purposes instead of any clinical benefits. In the present example, the content is to distract the user from an event such as a medical procedure. It is to be appreciated by a person of skill in the art with the benefit of this description that the manner by which the content is provided to the user is not particularly limited. In the present example, the output device 15 may provide virtual reality content to to the user. The manner by which virtual reality content is provided to a user is not limited and may involve a head mounted display having stereo projection capabilities. In addition, the output device 15 may further include motion detectors such as gyroscopes and accelerometers to detect and track head movement. By tracking head movement, images rendered by the output device 15 may be adjusted to allow the user to view different portions of the virtual reality by naturally moving their head. As an example, the output device 15 may include a commercially manufactured head mounted displace unit, such as the OCULUS RIFT.

The content provided is not particularly limited and may be selected from a library of content stored within the apparatus 10 or externally such as on an external server. Furthermore, it is to be appreciated that the different content may have different effects on different users due to individual differences. For example, some users may prefer a nature setting to achieve a calming effect while other users may prefer gaming content, such as driving a race car and competing with a simulation or other users connected by a network.

As a specific example, the content provider may be a UNITY 3D cross platform gaming engine. The gaming engine may receive data from the sensors 20 and transmit the data to an AZURE server. It is to be appreciated that the AZURE server may have a machine learning model deployed, which may be used to process the data and provide an estimate on stress of the user from whom the sensor 20 recorded data. This estimate may then be sent back to UNITY 3D cross platform gaming engine, which may also operate the content selection engine 30 to updates the game content provided to the user.

In another example, the output device 15 may provide augmented reality content to the user. In particular, the output device 15 may generate output images that include a background image with additional features superimposed or augmented. In another example, the output device 15 may include a clear screen to display features thereon while allowing the patient or user to look through the screen. Features may include characters or other objects may be superimposed onto a background image corresponding to the view behind the screen. As an example of an augmented reality device, the output device 15 may include a commercially manufactured head mounted displace unit, such as the MICROSOFT HOLOLENS.

Accordingly, the content may provide a feeling that the user is still within their present environment. Furthermore, the augmented reality may be combined with virtual reality hardware to provide a more immersive experience. The manner by which the output image is rendered is not particularly limited. In an example, apparatus may include a camera to capture the background image over which the features may be superimposed. The features are not particularly limited and may be provided by a content provider to provide a theme. For example, the theme may be a nature theme where features such as plants and calming wildlife may be superimposed on the background image of the clinical setting. Even within the theme, the content provided to the user may be adjusted to achieve a targeted amount of calming or distraction.

The manner by which the features are superimposed on the background is not particularly limited. For example, the apparatus 10 may include an augmented reality engine to analyze the background image and superimpose feature at appropriate locations on the such that the features are more seamlessly interwoven into the background image. In addition, the augmented reality engine may identify areas in the background image where the feature may be superimposed to blend in and appear to be part of the environment. For example, the augmented reality engine may identify empty areas in the background image, such as a blank space on a wall, or an open space on the floor. The augmented reality engine may then superimpose features such that they blend naturally into the environment. Continuing with the example above of a nature theme, the augmented reality engine may add some plants in the empty areas or add some calming wildlife.

As another example, the augmented reality engine may add features to improve the aesthetic appearance. Clinical settings may appear cold and have a lack of decor. Accordingly, the features such as artwork or printed images may be added. Furthermore, lighting may be changed from a bright white common in clinical settings to a softer color to provide additional calming effects.

In other examples, the output device 15 may be a screen to provide content such as video media. The video media may be content such as a movie for entertainment or educational purposes. Alternatively, the video media may also be interactive content to stimulate the user to provide further distraction and to reduce stress and anxiety.

The sensor 20 is to collect user data during the operation of the apparatus 10. In the present example, the sensor 20 is to measure a response to the output generated by the output device 15. The user data collected is not particularly limited and may include various data to provide information about the state of the user. For example, the user data may be physiological data to provide an indication of whether the user is stressed, or if the user is calm.

In an example, the sensor 20 may be a camera to collect an image of a facial expression of the user. The facial expression may provide indications of the state of the user. For example, images of the face of the user may be obtained and analyzed using facial recognition procedures. It is to be appreciated that a person of skill in the art with the benefit of this description that the manner by which the images are analyzed is not particularly limited. In the present example, the sensor 20 may be used to collect the images to be transmitted to an external analyzer for further processing to estimate the emotions, such as stress level or the level of engagement, of the user of the apparatus 10 while being provided with the content from the output device 15.

In other examples, the sensor 20 may be a biosensor used to detect muscular activity on the face of the user. In this example, the sensor 20 may include multiple electrical contact pads distributed at various regions of the face of the user where small electrical signals associated with the contraction of facial muscles may be detected. The electrical contact pads may be mounted on the output device 15 in examples where the output device 15 is to be mounted on the head of the user, such as for a virtual reality system. For example, muscles around the eyes of a use may be particularly indicative of the emotional state of the user. A smile accompanied by engagement of the muscles at the corners of the eyes may be interpreted as true smiles, as opposed to a smile that is put on voluntarily. Electrical signals near the eyes may also provide information about the eye such as gaze direction and movement which may be used as a substitute for optical methods of eye tracking. By tracking the eye motion, it may be possible to determine the level of engagement of the user with the content being generated by the output device 15.

In another example, the sensor 20 may be a heart rate monitor to measure the heart rate of the user. It is to be appreciated by a person of skill in the art with the benefit of this description that the heart rate of an individual may be indicative of the level of stress being experienced by the individual. The manner by which the sensor 20 measures the heart rate in the present example is not particularly limited. For example, the sensor 20 may measure the heart rate using electrical signals, such as with an electrocardiogram. In other examples, the sensor 20 may use use an optical system to detect blood flow in a nearby blood vessel. In additional to measuring the simple heart rate of a user, it is to be appreciated that other aspects of the heart rate may be measured, such as heart rate variability or regularity to assess the emotional state or stress level of the user. For example, various features of an electrocardiogram may be examined and the features of the heart beat as measured by the electrocardiogram may be examined to isolate various features, such as the average period of a beat, standard deviation of the period of the beat over a specified time interval or number of beats, the root mean squared of the periods of the beats over a specified time interval or number of beats, or the percentage of periods above or below a threshold value to infer and quantify the emotional state of the user. In other examples, the frequency domain features of the electrocardiogram may be examined and correlated with the emotional state of the user.

The communications interface 25 is to communicate over a network. In particular, the communications interface 25 may be connected to the sensor 20 to transmit the user data collected by the sensor 20 to an external analyzer (not shown). In the present example, the communications interface 25 may be to receive results from the analyzer, such as an assessment of the user data. In other examples, the communications interface 25 may also be used to transmit and receive data to other services, such as a content provider to request and receive additional content for the output device 15.

In the present example, the analyzer is not particularly limited and is to determine an objective measurement of the emotional state of the user based on the user data received. The manner by which the determination is made is not particularly limited. For example, the analyzer may compare data from the sensor 20 with data in a lookup table corresponding to a level of stress or level of engagement with the content provided by the output device 15. In other examples, the analyzer may use a machine learning or artificial intelligence model to determine the emotional state of the user based on the user data. The analyzer may be a separate stand-alone machine or may be part of the apparatus 10 in some examples. For example, the analyzer may be part of a server in communication with the apparatus 10 as well as other devices. In other examples, the analyzer may also be part of a cloud service operating over multiple distributed resources to provide a service for analyzing user data across a large geographical area.

The manner by which the communications interface 25 transmits and receives the data over a network is not limited and may include receiving an electrical signal via a wired connection. For example, the communications interface 25 may be network interface card to connect to the Internet. In other examples, the communications interface 25 may be a wireless interface to send and receive wireless signals, such as via a WiFi network. In other examples, the communications interface 25 may be to connect to another nearby device via a Bluetooth connection, radio signals or infrared signals from other nearby devices.

The content selection engine 30 is to control the content provided to the user based on an assessment of the stress level received from the analyzer. Accordingly, the content selection engine 30 may be used to monitor the stress level of the user or patient in the clinical setting to adjust the content provided to the user based on the reaction of the user. In a situation where the user finds the content to be difficult to understand or interact with, the user may become disengaged with the content. In such an example, the user may experience boredom and may ignore the content being provided via the output device 15. If the content provided is to be ignored, any beneficial effects of the apparatus 10 may not be felt by the user. Accordingly, the stress level of the user may then increase as the user becomes more focused on the clinical setting and approach the same level of stress as if the device were not used. Accordingly, in such a situation, the content selection engine 30 may alter the content provided to the user by selecting content considered to be more entertaining by the user or to decrease the level of difficult of the interactive content. Alternatively, if the content provided is too stimulating, the stress level of the user may further be increased due to sensory overload or cognitive overload. Accordingly, the content may have negative effects for the user. In such a situation, the content selection engine 30 may automatically alter the content provided to the user by selecting content considered to be more calming by the user or to increase the level of difficult of the interactive content to promote more interest and engagement by the user or patient, which thus provides a distraction for the user from the clinical setting.

The manner by which the content selection engine 30 changes the content is not particularly limited. For example, the response to content by individual users may be different depending on the age or personal preferences of the user. Accordingly, the content selection engine 30 may initially select content to be displayed to the user in a random manner. Upon providing the content to the user, the sensors 20 may be used to measure the reaction which is determined by the analyzer. Based on the analysis received from the analyzer, the content selection engine 30 may modify the content and the sensor 20 measure the reaction from the user after each modification. In other examples, the sensor 20 may be continuously operating and measuring user data continuously regardless of whether a modification has been made. Therefore, content considered to be sufficiently calming to the user or patient may be selected through an iterative process. The modifications to the content are not limited and may include subtle changes or complete changes based on the strength of the reaction from the user. The selection process and history may be associated with a profile of the specific user or patient and subsequently used to select content for other similar users or patients to reduce the efforts to determine the appropriate content. In other examples, the selection process may be stored in a dataset for training the content selection engine 30 using a machine learning process. In examples where the content may be in an interactive game, the content selection engine 30 may be used to modify the level of difficult of the game. For example, if the user is disengaging from the game because it is too difficult such that the user continually fails, the content selection engine 30 will decrease the level of difficulty. Alternatively, if the user is finding the game too simple and may successfully complete the tasks in the game quickly with little effort, the content selection engine 30 may increase the level of difficulty of the interactive game.

In other examples, the content selection engine 30 may select the content based on prior data such as data from the user or patient. For example, the user or patient may be asked to complete a survey prior to entering the clinical setting that may indicate preferences. The preferences may be used to select the initial content as well as to determine what modifications are to be subsequently made in order to achieve a sufficient level of calming to offset the stress induced by being in a clinical setting.

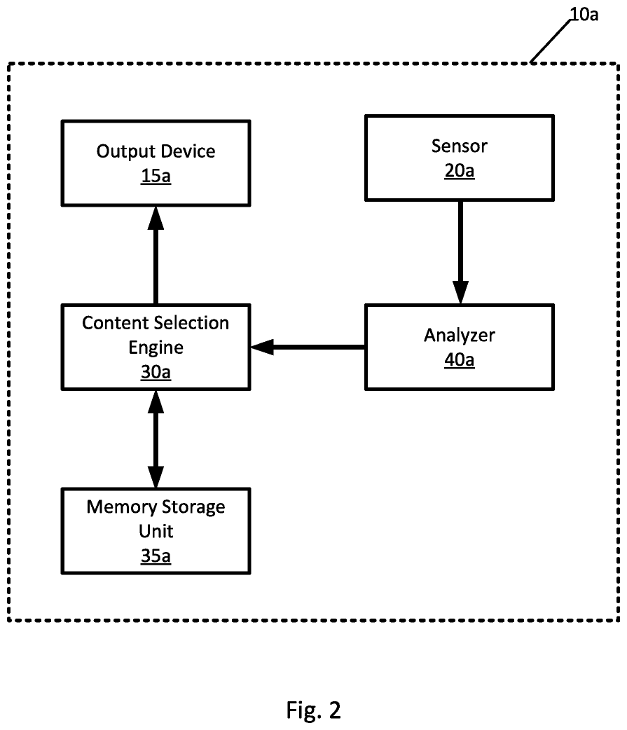

Referring to FIG. 2, another example of an apparatus to manage stress in a clinical setting is shown at 10a . Like components of the apparatus 10a bear like reference to their counterparts in the apparatus 10, except followed by the suffix “a”. The apparatus 10a includes an output device 15a , a sensor 20a , a content selection engine 30a , a memory storage unit 35a , and an analyzer 40a . Although the present example shows the content selection engine 30a and the analyzer 40a as separate components, in other examples, the content selection engine 30a and the analyzer 40a may be part of the same physical component such as a microprocessor configured to carry out multiple functions.

It is to be appreciated that in the present example, the output device 15a and the sensor 20a function in a substantially similar manner as the output device 15 and the sensor 20 in the example above. For example, the output device 15a and the sensor 20a may be similar or identical to the output device 15 and the sensor 20 described above in the previous example. Furthermore, it is to be appreciated that the present example may not include a communications interface. Accordingly, the apparatus 10a may be used as a standalone device without connecting to a network.

The memory storage unit 35a may be to store content to be provided to the user or patient in the clinical setting. In the present example, the memory storage unit 35a may be in communication with the content selection engine 30a . Upon selecting the content to be provided to the user, the output device 15a may retrieve the content from the memory storage unit 35a to be provided to the user. In the present example, the content stored on the memory storage unit 35a may be stored in a database accessible by the output device 15a and the content selection engine 30a . In particular, the memory storage unit 35a may maintain a library of content from which the content selection engine 30a may select and from which the output device 15a may retrieve and render content data. The manner by which the content is stored on the memory storage unit 35a is not limited and may include storing content in a database having an index. The content may also be sorted within the database for faster retrieval by the content selection engine 30a . For example, the content may be sorted by genre, title, author name, producer, or date of creation. As another example, the content may be sorted using an index specific to a user or patient based on a predetermined response to the content. Accordingly, in such an example, the content selection engine 30a may provide customized adjustments of the content provided to the user or patient from the memory storage unit 35a based on the stress level of the user or patient.

In the present example, the memory storage unit 35a is not particularly limited and may include a non-transitory machine-readable storage medium that may be any electronic, magnetic, optical, or other physical storage device. The memory storage unit 35a may be loaded with content via a communications interface (if present), or by directly transferring the content from a portable memory storage device connected to the apparatus, such as a memory flash drive. In other examples, the memory storage unit 35a may be an external unit such as an external hard drive, or a cloud service providing content.

It is to be appreciated by a person of skill in the art with the benefit of this description that the memory storage unit 35a may also be used to store additional information such as data captured by the sensor 20a prior to being processed by the analyzer 40a as well as the results from the analyzer 40a.

In addition, the memory storage unit 35a may be used to store instructions for general operation of the apparatus 10a . In particular, the memory storage unit 35a may also store an operating system that is executable by a processor to provide general functionality to the apparatus 10a , for example, functionality to support various applications. The memory storage unit 35a may additionally store instructions to operate the content selection engine 30a and the analyzer 40a . Furthermore, the memory storage unit 35a may also store hardware drivers to communicate with other components and other peripheral devices of the apparatus 10a , such as the output device 15a , the sensor 20a , and the analyzer 40a as well as various other additional output and input devices (not shown).

In the present example, the analyzer 40a is not particularly limited and is to provide an objective measurement of the emotional state or stress level of the user based on the user data received via the sensor 20a . The manner by which the measurement is carried out is not particularly limited. For example, the analyzer 40a may compare data from the sensor 20a with data in a lookup table stored on the memory storage unit 35a . The data measured by the sensor 20a may correspond to a level of stress listed on the lookup table. In other examples, the analyzer 40a may use a machine learning or artificial intelligence model to determine the emotional state and stress level of the user based on the user data.

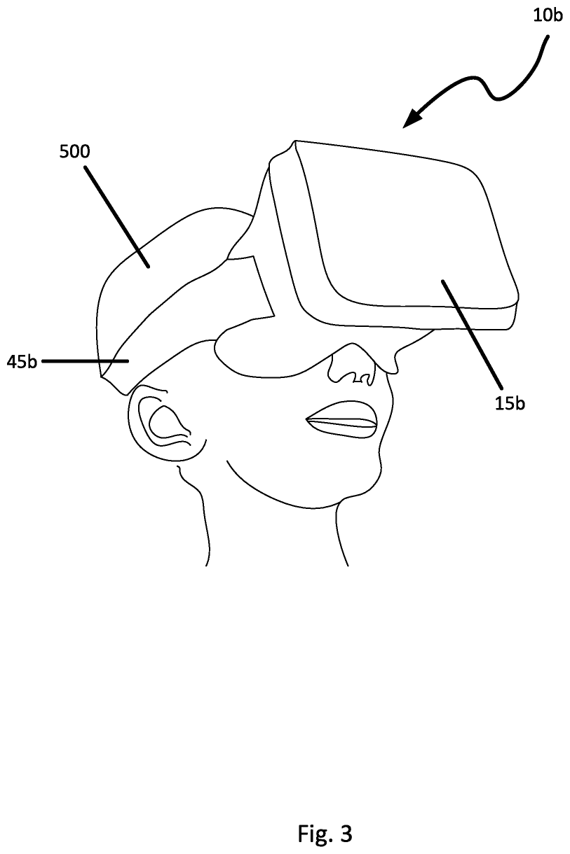

Referring to FIG. 3, another example of an apparatus to manage stress in a clinical setting is shown as 10b in operation with a user 200. Like components of the apparatus 10b bear like reference to their counterparts in the apparatus 10, except followed by the suffix “b”. The apparatus 10b includes an output device 15b and a mounting mechanism 45b . It is to be appreciated by a person of skill in the art with the benefit of this description that the apparatus 10b may be similar or identical to one of the apparatus 10a or the apparatus 10b . In other examples, the apparatus 10b may be another variant. Accordingly, the apparatus 10b may also include a sensor (not shown) and a content selection engine (not shown).

The mounting mechanism 45b is to mount the apparatus 10b on the user 200. By mounting the apparatus 10b directly on the user, such as over a sensory receptors, the apparatus 10b may be used to provide a personal experience to the user 200. In the present example, the mounting mechanism 45b is a flexible biasing element, such as a band to secure the apparatus 10b on the head of the user such that the output device 15b is positioned in front of the eyes of the user 200 to provide visual images visible by the user 200 and not by other individuals in the proximity of the user 200. In other examples, the output device 15b may be configured to provide sounds for the user 200. Accordingly, the mounting mechanism 45b may be modified to secure the output device 15b over the ears of the user 200 to provide a personal audio experience by not allowing other individuals within proximity of the user 200 to overhear any audio. In further examples, the output device 15b may provide both audio and video output such that both the ears and the eyes of the user 200 are to be covered and insulated from external sounds and visual distractions.

Although the mounting mechanism 45b shown in FIG. 3 is a band made from an elastic material attached to the output device 15b , the mounting mechanism 45b is not limited. In other examples, the mounting mechanism 45b may be a hat or helmet to be placed over the head of the user 200. As another example, the mounting mechanism 45b may also include a contour or other physical feature to mate with features on the face of the user 200. Further examples may include various braces and straps to secure the apparatus 10b to the head of the user 200.

The format of the personal experience provided by the apparatus 10b is not particularly limited. For example, the apparatus may provide a virtual reality experience. In particular, the virtual reality experience may involve providing stereo images with the output device 15b to the user 200 to simulate stereo vision and provide depth perception. In addition, the apparatus 10b may further include motion detectors such as gyroscopes and accelerometers to detect and track movements of the user 200 to adjust the images provided by the output devices 15b to allow the user to view different portions of the virtual reality my naturally moving their head. By providing content via the head mounted apparatus 10b , it is to be appreciated that each individual user 200 may be provided with unique experiences to achieve the desired calming effect for the user 200.

In another example, the apparatus 10b may provide an augmented reality experience. In particular, the augmented reality experience may involve providing stereo images with the output device 15b to the user 200 to provide a background image with additional features superimposed or augmented thereon. In addition, the apparatus 10b may further include a camera to obtain the background image onto which the features are to be superimposed. By providing content via the head mounted apparatus 10b , it is to be appreciated that each individual user 200 may be provided with a unique augmented reality to achieve the desired calming effect for the user 200.

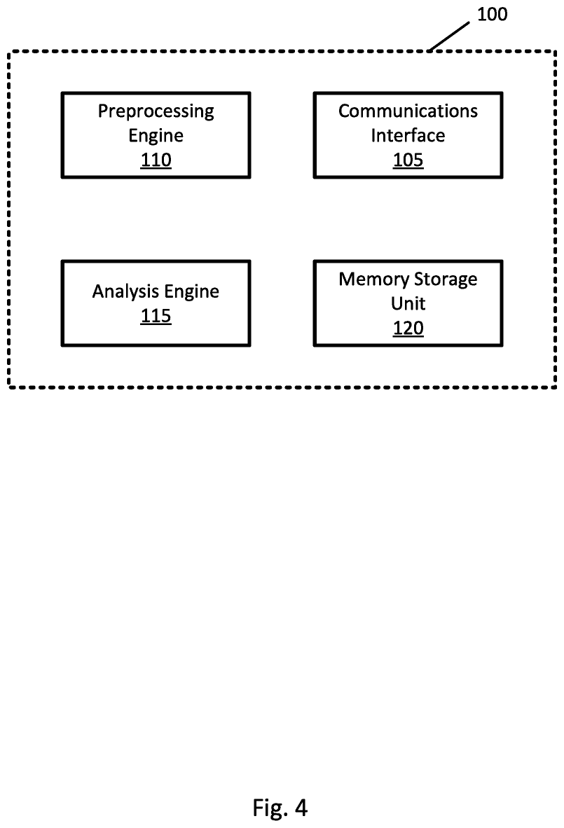

Referring to FIG. 4, an example of a server 100 which may be used to process data from the apparatus 10 to determine and provide a stress level of a user based on data collected from the sensor 20 is generally shown. The server 100 is not particularly limited and may be any computing device capable of processing data received from the apparatus 10. For example, the server 100 may be a traditional server machine or a desktop computer. In other examples, the server 100 may be a tablet, or a smartphone if the computational demands may be met by such devices. The server 100 may also include additional components, such as interfaces to communicate with other devices, and may include peripheral input and output devices to interact with an administrator of the server 100. In the present example, the server 100 includes a communications interface 105, a preprocessing engine 110, an analysis engine 115, and a memory storage unit 120.

The communication interface 105 is to communicate with the apparatus 10 over a network. In the present example, the server 100 may be a cloud server to be managed by the apparatus 10. Accordingly, the communication interface 105 may be to receive data from the apparatus 10 and to transmit results, such as a quantified measure of the stress level of a user or another assessment of the user data, back to the apparatus 10 after processing the data. The manner by which the communication interface 105 receives and transmits the data is not particularly limited. In other examples, the communications interface 105 may also be used to transmit and receive data for other services, such as to receive requests for content and to provide content to the apparatus 10. In the present example, the server 100 may connect with the apparatus 10 at a distant location over a network, such as the internet. In other examples, the communication interface 105 may connect to the apparatus 10 via a local connection, such as over a private network or wirelessly via a Bluetooth connection. In the present example, the server 100 may be a central server connected to multiple apparatuses either within a similar geographical region or over a wide area. For example, the server 100 may be connected via a local network to multiple apparatuses within a clinical setting, such as in a hospital. In this example, the server 100 may be operated by operator of the clinical setting, such as a hospital, to provide the benefits to multiple users having individual reactions to the content provided by each apparatus 10. In another example, the server 100 may be a virtual server existing in the cloud where functionality may be distributed across several physical machines to multiple clinical settings and provided as a fee-for-service to each of the clinical settings.

The preprocessing engine 110 may be to carry out the initial analysis of the data received from the apparatus 10. In the present example, the preprocessing engine 110 may be used to prepare the data from the apparatus 10 for the analysis engine 115. It is to be appreciated by a person of skill in the art with the benefit of this description that the preprocessing engine 110 is not particularly limited and may carry out different functions depending on the type of data received from the apparatus 10 and the type of analysis to be carried out. For example, if the data received from the apparatus 10 includes images of the face of a user for image analysis to determine a stress level, the preprocessing engine 110 may be used to extract features for further analysis. The preprocessing engine 110 may identify features such as the eyes or mouth which may be subsequently analyzed by the analysis engine 115 to determine the emotional state of the user. In another example, if the data received from the apparatus 10 includes biosensor data to detect muscular activity, the preprocessing engine 110 may be used to isolate the muscular activities based on location on the face, strength, or other factors for subsequent processing by the analysis engine 115.

In other examples, the data received by the preprocessing engine 110 may include user data of multiple types from multiple modalities. For example, one modality may be heart rate data from an electrocardiogram, and another modality may be a facial expression or hand gesture in the form of video or images. The multiple types of data may be measured using the sensor 20 or multiple sensors. In the present example, the preprocessing engine 110 may separate the modalities and preprocess the data separately for subsequent processing by the analysis engine 115. In other examples, the server 100 may include a plurality of of preprocessing engines where each preprocessing engine is to preprocess a specific type of data received from the apparatus 10.

The analysis engine 115 is to analyze the data to determine a stress level of the user. In the present example, the analysis engine 115 may receive the preprocessed data from the preprocessing engine 110. By analyzing the preprocessed data, it is to be appreciated that the computational resources used by the analysis engine 115 may be reduced. In other examples, the analysis engine 115 may be used to analyze the raw data received from the apparatus 10, such as in examples where the preprocessing engine 110 is omitted.

In the present example, the analysis engine 115 is to analyze the data using a convolutional neural network model to identify the emotional state of the user, such as the stress level. The manner by which the convolutional neural network model is applied is not limited and may be dependent on the type of data received at the analysis engine 115. It is to be appreciated that the emotional state of a person may be determined based on various cues and that several different features or gestures may be be used to make a determination. For example, the facial expression of a user, hand gestures made by the used, a heart rate, electrochemical activity in the brain, speech, such as tone, stuttering, and choice of language, etc. may all be used to assess the emotional state of a user. In other examples where the user is providing continuous input, such as during an interaction with gaming content, the input may be analyzed to determine the emotional state of the user as well as the level of engagement with the game. For example, the convolutional neural network may be used to analyze images of facial features to determine if an expression indicates the user or patient is nervous, such as raised eyebrows, mouth slightly ajar, or wide eyes, correspond to nervousness or stress in the clinical setting. In another example, the convolutional neural network may be used to analyze speech where the apparatus 10 measures audio signals. In yet another example, the convolutional neural network may be used to analyze a heartbeat and or breathing to determine any irregularities or speed. Accordingly, the analysis engine 115 may identify the emotional state of a user and assign an arbitrary index value. The index value may then be used to assess the state of the user and provided to the apparatus 10 where the apparatus 10 may make adjustments to the content provided to the user or patient.

In examples where multiple modalities of preprocessed data are received from the preprocessing engine 110, the analysis engine 115 may apply a multimodal convolutional neural network to the preprocessed data received from the preprocessing engine 110. The manner by which the analysis engine 115 handles the data is not particularly limited. For example, the preprocessed data may be received in a single format which allows the multimodal convolutional neural network to analyze the data as a whole to reduce potential noise based on the multiple types of data. Accordingly, in this example, the multimodal convolutional neural network may be pretrained to analyze the multimodal data received at the analysis engine 115. It is to be appreciated that the analysis engine 115 may also carry out temporal data alignment between the modalities as well as data transformations. In other examples, the server 100 may include a plurality of of analysis engines where each analysis engine is to analyze the different modalities separately. The results may then be subsequently compared and verified against each other.

The manner by which the analysis engine 115 is trained is not particularly limited. For example, training data available from other sources may be used to train the analysis engine 115. In such an example, the training data may be purchased from a provider or obtained by carrying out research and generating a test data set. In other examples, the analysis engine 115 may continuously learn from data collected and analyzed during operation. In this example, the data received from the apparatus 10 may be stored in the memory storage unit 120 as well as the results of the processing for periodic retraining of analysis engine 115. The frequency at which the analysis engine 115 is retrained is not limited and may be dependent on various factors such as the amount of computation resources available, which may include network latencies where data is to be downloaded from other sources, or in processor availability. In some examples, the retraining may occur weekly, daily, or hourly. In other examples, the retraining may occur more frequently to approach real-time retraining. Alternatively, some examples may not retrain the convolutional neural network automatically and carry out the process upon administrator intervention.

The process by which the analysis engine 115 carries out machine learning is not particularly limited and may involve using commercial or open source machine learning processes. As a specific example of the analysis engine 115 carrying our machine learning, it may be assumed that tools such as the Tree-Based Pipeline Optimization Tool (TPOT) are used. In the present example, a supervised machine learning model is used where the training dataset includes a one-to-one correspondence between the content, the user data, and emotional state or the stress level of the user. The stress level of the user may be determined and associated with the user data based on various tests carried out in a neutral setting. For example, a baseline measurement of the user data may be recorded without any stimulus outside of the clinical setting to obtain exemplary user data for an unstressed individual. Content may then be provided to the user to measure a response. The content is not particularly limited and may include content that provides acute stress, such as a roller coaster simulation. Cognitive stress may also be measured by providing the user with a psychological test, such as the Stroop Test. In addition, other stress may be measure with content such as a game, for example, Tetris, bubble bloom, or pong breakout.

The memory storage unit 120 is to store data that may be generated or used by the server 100. In the present example, the memory storage unit 120 may include a non-transitory machine-readable storage medium that may be any electronic, magnetic, optical, or other physical storage device. The memory storage unit 120 may also be used maintain an operating system to operate the server 100. Furthermore, the memory storage unit 120 may be used to store content to be distributed to the apparatus 10 upon request as well as training data to train the analysis engine 115.

Variations to the server 100 are contemplated. For example, it is to be appreciated that in some examples, the server 100 may also include a content selection engine. Accordingly, in such an example, the server 100 may receive raw data from the apparatus 10, process the raw data using the preprocessing engine 110 and the analysis engine 115 to determine the emotional state of the user of the apparatus 10. Based on the emotional state of the user, the server 100 may control the content being provided for output at the apparatus 10 to calm the user or patient in the clinical setting.

Furthermore, it is to be appreciated that although the above examples discussed involve having the server 100 communicate with the apparatus 10, substitutions may be made. For example, the server 100 may be to communicate with the apparatus 10a to serve as a content provider or to provide additional analysis capabilities to the analyzer 40a . In particular, since the apparatus 10a is intended to be a personal device that is physically smaller, the analyzer 40a may not have sufficient resources. Similarly, the server 100 may be in communication with the apparatus 10b to provide analysis of the data collected at the apparatus 10b as well as to provide content to the apparatus 10b upon request.

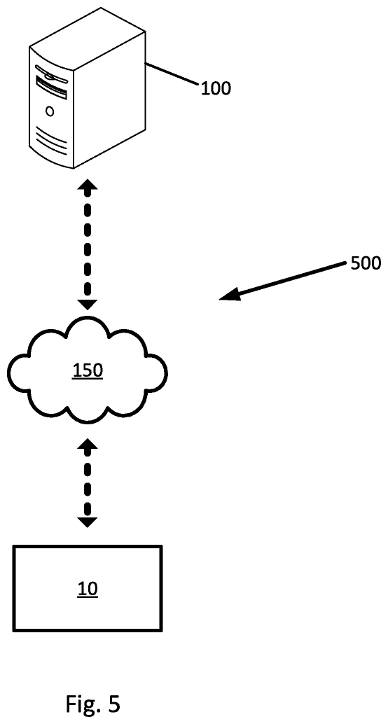

Referring to FIG. 5, an example of a system to manage stress in a clinical setting is generally shown at 500. In the present example, the apparatus 10 is in communication with a the server 100 via the network 150. In this example, the network 150 may be any type of communications network to connect electronic devices. For example, the network 150 may be a local network that is either wired or wireless. In other examples, the network 150 may be the Internet for connecting devices across greater distances using existing infrastructure. Furthermore, it is to be understood that the server 100 may be connected to multiple apparatuses where the server is to process data and/or provide content to the users.

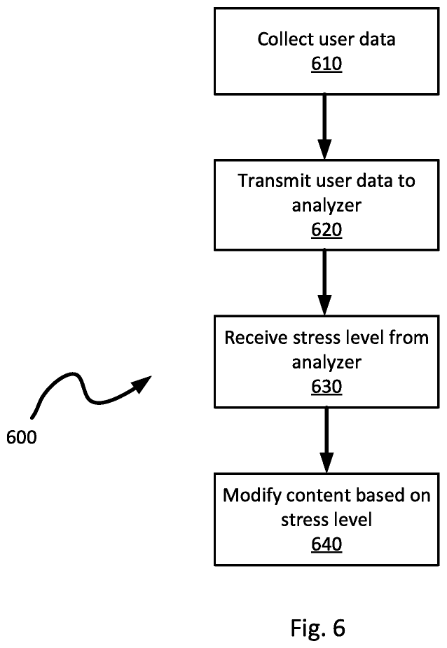

Referring to FIG. 6, a flowchart of an example method manage stress in a clinical setting is generally shown at 600. In order to assist in the explanation of method 600, it will be assumed that method 600 may be performed with the apparatus 10. Indeed, the method 600 may be one way in which apparatus 10 along with the server 100 may be configured. Furthermore, the following discussion of method 600 may lead to a further understanding of the system 500. In addition, it is to be emphasized, that method 600 may not be performed in the exact sequence as shown, and various blocks may be performed in parallel rather than in sequence, or in a different sequence altogether.

Beginning at block 610, user data is collected using the sensor 20. In the present example, the user data may be a reaction to content provided to a user via the output device 15. The user data collected is not particularly limited and may include various data to provide information about the state of the user. For example, the user data may be physiological data to provide an indication of whether the user is stressed, or if the user is calm. For example, the sensor 20 may collect an image of a facial expression of the user, which may be used to determine the state of the user. For example, portions of one or more images of the face of the user may be subsequently processed using facial recognition methods.

Next at block 620, the user data is transmitted to be processed by an analyzer. The analyzer is not particularly limited and may be a local analysis engine where the transmission of the user data is an internal process to transfer the data from a sensor to the analysis engine, such as in the case of the apparatus 10a having the analyzer 40a . In other examples, the analysis engine may be on a separate device such that the user data is to be transmitted across greater distances. For example, the user data may be transmitted to a server 100 located remotely or in the cloud to carry out the analysis of the user data. It is to be appreciated that the manner by which the images are analyzed is not particularly limited.

Block 630 involves receiving a stress level from the analyzer to which the user data was transmitted above in block 620. The stress level received from the analyzer may be indicative of the emotional state of the user. In the present example, the analysis engine may identify the emotional state of a user and assign an arbitrary index value. The index value may then be used to assess the state of the user and provided to the apparatus 10 where the apparatus 10 may make adjustments to the content provided to the user or patient. In the present example, the index value may rank the level of stress of the user. In other examples, the index value may be used to quantitatively describe the level of engagement of the user. In further examples, multiple index values may be used to measure various aspects of the user.

In the present example, block 640 involves modifying the content provided to the user based on a stress level as determined in block 630. In the present example, the content provided to the user via the output device 15 may be modified by the content selection engine 30 based on the values received from block 430. The manner by which the content selection engine 30 changes the content is not particularly limited. For example, the response to content by individual users may be different depending on the age or personal preferences of the user. Upon initiating treatment with the apparatus 10, the content selection engine 30 may select content to be displayed to the user in a random manner or based on the known characteristics of the user, such as age and/or interests. Upon providing the content to the user, the content selection engine 30 may receive information from an analyzer based on data collected from the user via the sensor 20. Based on the values received at block 630, the content selection engine 30 may be used to modify the content and subsequently monitor the reaction from the user based on additional data received on new user data after the content has been modified. The modifications to the content are not limited and may include subtle changes or complete changes based on the results received at block 630. For example, the content may be modified to add additional calming features, such as changing the lighting or audio. In other examples, the content may be completely changed to provide a different theme altogether.

While specific examples have been described and illustrated, such examples should be considered illustrative only and should not serve to limit the accompanying claims.

What is claimed is:

1. An apparatus comprising:
an output device to provide content to a user, wherein the content is to distract the user from an event;
a sensor to collect user data in response to the content generated by the output device, wherein the user data is to provide information about a state of the user;
a communications interface connected to the sensor, the communication interface to transmit the user data to an analyzer, wherein the analyzer is to determine a stress level of the user based on the user data, the communications interface further to receive the stress level of the user data from the analyzer; and
a content selection engine to control the content provided to the user based on the stress level.

2. The apparatus of claim 1, further comprising a mounting mechanism to mount on the user to provide a personal experience.

3. The apparatus of claim 2, wherein the personal experience is a virtual reality experience.

4. The apparatus of claim 2, wherein the personal experience is an augmented reality experience.

5. The apparatus of claim 1, further comprising a memory storage unit to store the content to be provided to the user via the output device.

6. The apparatus of claim 5, wherein the memory storage unit is to maintain a library of content from which the content selection engine is to select the content.

7. The apparatus of claim 1, wherein the user data is physiological data.

8. The apparatus of claim 7, wherein the physiological data is a facial expression, and the sensor is a camera to collect an image of the facial expression.

9. The apparatus of claim 7, wherein the physiological data is a heart rate, and the sensor is a heart rate monitor.

10. A server comprising:
a communications interface to communicate with a network, the communication interface to receive user data from a client device, wherein the client device includes a sensor to collect the user data;
a preprocessing engine to receive the user data from the communications interface, the preprocessing engine to carry out an initial analysis of the user data to generate preprocessed data;
an analysis engine to receive the preprocessed data, the analysis engine to analyze the preprocessed data to estimate a stress level of a user using a machine learning model; and
a memory storage unit to store results from the analysis engine for use as training data for the machine learning model.

11. The server of claim 10, wherein the user data is physiological data.

12. The server of claim 11, wherein the physiological data is a facial expression, and the sensor is a camera to collect an image of the facial expression.

13. The server of claim 11, wherein the physiological data is a heart rate, and the sensor is a heart rate monitor.

14. A non-transitory machine-readable storage medium encoded with instructions executable by a processor, the instructions to direct the processor to:
to collect user data using a sensor in response to content generated by an output device, wherein the user data is to provide information about a state of the user;
to transmit the user data to an analyzer, wherein the analyzer is to determine a stress level of the user based on the user data;
to receive the stress level of the user data from the analyzer; and
to modify the content provided to the user based on the stress level.

15. The non-transitory machine-readable storage medium of claim 14, wherein the instructions direct the processor to provide the content to a user to distract the user from an event.

16. The non-transitory machine-readable storage medium of claim 15, wherein the instructions direct the processor to provide a virtual reality experience.

17. The non-transitory machine-readable storage medium of claim 14, wherein the instructions direct the processor to store the content to be provided to the user via the output device in a memory storage unit for subsequent retrieval.

18. The non-transitory machine-readable storage medium of claim 17, wherein the instructions direct the processor to maintain a library of content from which select the content.

19. The non-transitory machine-readable storage medium of claim 14, wherein the instructions direct the processor to collect physiological data, wherein the physiological data is a facial expression.

20. The non-transitory machine-readable storage medium of claim 14, wherein the instructions direct the processor to collect heart rate data with a heart rate monitor.
